(19) United States
(12) Patent Application Publication
(10) Pub. No.: US 2026/0165722 A1
(43) Pub. Date: Jun. 18, 2026

(54) Coracoid Clamp

(71) Applicant: Stryker Corporation, Portage, MI (US)

(72) Inventor: Jeremy Graul, Denver, CO (US)

(73) Assignee: Stryker Corporation, Portage, MI (US)

(21) Appl. No.: 19/413,731

(22) Filed: Dec. 9, 2025

Related U.S. Application Data
(60) Provisional application No. 63/734,546, filed on Dec. 16, 2024.

(51) Int. Cl. A61B 17/28; A61B 17/00; A61B 17/17; A61B 17/56

(52) U.S. Cl. CPC A61B 17/282; A61B 17/1778; A61B 17/2833; A61B 17/56; A61B 2017/00407; A61B 2017/2808; A61B 2017/2837; A61B 2017/564

(57) ABSTRACT

A coracoid clamp and related methods for preparing a bone block. The coracoid clamp includes first and second elongate arms, each with a proximal handle portion and a distal clamping portion. The distal clamping portion of the first elongate arm defines a first bone block engagement structure configured to engage a planar surface of a bone block. The distal clamping portion of the second elongate arm defines a second bone block engagement structure configured to engage a non-planar surface of the bone block opposite the planar surface. The first and second distal clamping portions are pivotable towards each other to engage the bone block with the first and second bone block engagement structures. The distal clamping portion of the first elongate arm defines lumens with a circular transverse cross-sectional shape, and the distal clamping portion of the second elongate arm defines a lumen with an elongated transverse cross-sectional shape.

### CROSS-REFERENCE TO RELATED APPLICATION

This application claims priority to U.S. Provisional Patent Application No. 63/734,546, entitled “Coracoid Clamp,” filed on Dec. 16, 2024, the entire disclosure of which is hereby incorporated by reference.

### BACKGROUND

The Latarjet procedure is a surgical technique used to treat shoulder instability. An important step in this procedure involves the preparation of a bone block, such as one harvested from the patient's coracoid, for fixation to the patient's glenoid. Conventional methods for preparation of the bone block involve stabilizing a portion of the patient's coracoid with a clamp and drilling holes through the coracoid freehand. However, this approach can have an increased risk of inaccurate alignment of the drill holes relative to a prepared flattened surface of the coracoid to be secured against the glenoid. Such misalignment may result in rotational instability of the coracoid graft and potential non-union with the glenoid, compromising the success of the procedure.

A need therefore exists in the art for an improved tool that helps ensure accurate alignment of drill holes formed in a bone block.

### SUMMARY

In one exemplary aspect, a coracoid clamp includes a first elongate arm including a first proximal handle portion and a first distal clamping portion. The first distal clamping portion includes a first side and a second side opposite the first side, the first side of the first distal clamping portion defining a first bone block engagement structure configured to engage a planar surface of a bone block. The coracoid clamp also includes a second elongate arm including a second proximal handle portion and a second distal clamping portion. The second distal clamping portion includes a first side and a second side opposite the first side, the first side of the second distal clamping portion defining a second bone block engagement structure configured to engage a non-planar surface of the bone block opposite the planar surface. The first elongate arm is pivotally coupled to the second elongate arm such that the first and second distal clamping portions are pivotable towards each other to engage the bone block with the first bone block engagement structure and the second bone block engagement structure. The first distal clamping portion defines a plurality of first lumens each extending through the first bone block engagement structure and the second side of the first distal clamping portion, each of the first lumens having a circular cross sectional shape in a direction transverse to a longitudinal axis of the first lumen. The second distal clamping portion defines a second lumen extending through the second bone block engagement structure and the second side of the second distal clamping portion, the second lumen having an elongated cross sectional shape in a direction transverse to a longitudinal axis of the second lumen.

In another exemplary aspect, the coracoid clamp includes a first elongate arm including a first proximal handle portion and a first distal clamping portion. The first distal clamping portion includes a first side and a second side opposite the first side, the first side of the first distal clamping portion defining a first bone block engagement structure configured to engage a planar surface of a bone block. The coracoid clamp also includes a second elongate arm including a second proximal handle portion and a second distal clamping portion. The second distal clamping portion includes a first side and a second side opposite the first side, the first side of the second distal clamping portion defining a second bone block engagement structure configured to engage a non-planar surface of the bone block opposite the planar surface. The first elongate arm is pivotally coupled to the second elongate arm such that the first and second distal clamping portions are pivotable inwards towards each other with the first side of the first distal clamping portion defining an inward facing side of the first distal clamping portion and the second side of the first distal clamping portion defining an outward facing side of the first distal clamping portion. The first distal clamping portion defines a lumen extending through the first bone block engagement structure and the second side of the first distal clamping portion. The first bone block engagement structure includes a plurality of first teeth each extending inwards to define a free end, the free ends of the first teeth being configured to engage the planar surface of the bone block. The first side of the first distal clamping portion also defines a lip distal the first bone block engagement structure. The lip likewise extends inwards to define a free end, the free end of the lip being located further inwards than the free end of each of the first teeth.

In another exemplary aspect, a method of preparing a bone block during a Latarjet procedure using a coracoid clamp includes providing a coracoid clamp. The coracoid clamp includes a first elongate arm including a first proximal handle portion and a first distal clamping portion, the first distal clamping portion including a first side and a second side opposite the first side, and the first side of the first distal clamping portion defining a first bone block engagement structure configured to engage a planar surface of the bone block. The coracoid clamp also includes a second elongate arm including a second proximal handle portion and a second distal clamping portion, the second distal clamping portion including a first side and a second side opposite the first side, the first side of the second distal clamping portion defining a second bone block engagement structure configured to engage a non-planar surface of the bone block that is opposite the planar surface. The first elongate arm is pivotally coupled to the second elongate arm such that the first and second distal clamping portions are pivotable towards each other, the first distal clamping portion defines a plurality of lumens each extending through the first bone block engagement structure and the second side of the first distal clamping portion, and the first side of the first distal clamping portion defines a lip distal to the first bone block engagement structure. The method also includes positioning the first distal clamping portion on a bone block such that the first bone block engagement structure sits on the planar surface of the bone block and the lip is positioned against a side of the bone block that extends from the planar surface and is adjacent a conjoint tendon. The method also includes engaging the coracoid clamp to urge the second distal clamping portion towards the first distal clamping portion to engage the non-planar surface of the bone block with the second bone block engagement structure and hold the bone block between the first bone block engagement structure and the second bone block engagement structure. The method also includes drilling a hole into the held bone block through each of the lumens.

Other objects, features and advantages of the present invention will be readily appreciated as the same becomes better understood after reading the subsequent description taken in connection with the accompanying drawings.

### BRIEF DESCRIPTION OF THE DRAWINGS

Advantages will be readily appreciated as the same becomes better understood by reference to the following detailed description when considered in connection with the accompanying drawings, wherein:

FIG. 1 is a perspective view of a coracoid clamp including first and second distal clamping portions pivotable inwards towards one another to engage a bone block for drilling according to an exemplary aspect.

FIG. 2 is a perspective view of the first distal clamping portion including a first bone block engagement structure configured to engage a planar surface of a bone block according to an exemplary aspect.

FIG. 3 is a perspective view of the second distal clamping portion including a second bone block engagement structure configured to engage a non-planar surface of a bone block according to an exemplary aspect.

FIGS. 4A and 4B are perspective views of the first and second distal clamping portions being pivoted to a first position and a second position according to an exemplary aspect.

FIG. 5 is a flowchart of a method for preparing a bone block for a Latarjet procedure using the coracoid clamp of FIG. 1 according to an exemplary aspect.

FIG. 6 is a perspective view of the first bone block engagement structure of the first distal clamping portion of the coracoid clamp of FIG. 1 having been positioned against a planar surface of a bone block according to an exemplary aspect.

FIG. 7 is a perspective view of the first and second distal clamping portions of the coracoid clamp of FIG. 1 having been urged towards each other so that a bone block is engaged between the first and second bone block engagement structures of the coracoid clamp according to an exemplary aspect.

FIG. 8 is a front perspective view of the first and second distal clamping portions of the coracoid clamp of FIG. 1 having been urged towards each other so that a bone block is engaged between the first and second bone block engagement structures of the coracoid clamp while a coracoacromial ligament extends laterally from the bone block according to an exemplary aspect.

FIG. 9 is a perspective view of the first and second distal clamping portions of the coracoid clamp of FIG. 1 having been urged towards each other so that a bone block is engaged between the first and second bone block engagement structures of the coracoid clamp and a drill bit having been passed through lumens formed in the first and second distal clamping portions of the coracoid clamp to drill a hole into the engaged bone block according to an exemplary aspect.

### DETAILED DESCRIPTION

The present application describes coracoid clamps generally designed to aid in the precise drilling of holes in a bone block for use in a Latarjet procedure. In one aspect, a coracoid clamp may include a first bone block engagement structure specifically configured to engage a prepared planar surface of the bone block and a second bone block engagement structure specifically configured to engage a natural curvature of the bone block that is opposite the planar surface. The interaction of the first bone block engagement structure with the planar surface of the bone block while the bone block is being held between the first and second bone block engagement structures helps prevent rotation and maintain stability of the bone block during drilling. These and other features of the coracoid clamp described herein provide a significant improvement over conventional methods, offering enhanced stability, precision, and reliability in the preparation of a bone block during the Latarjet procedure.

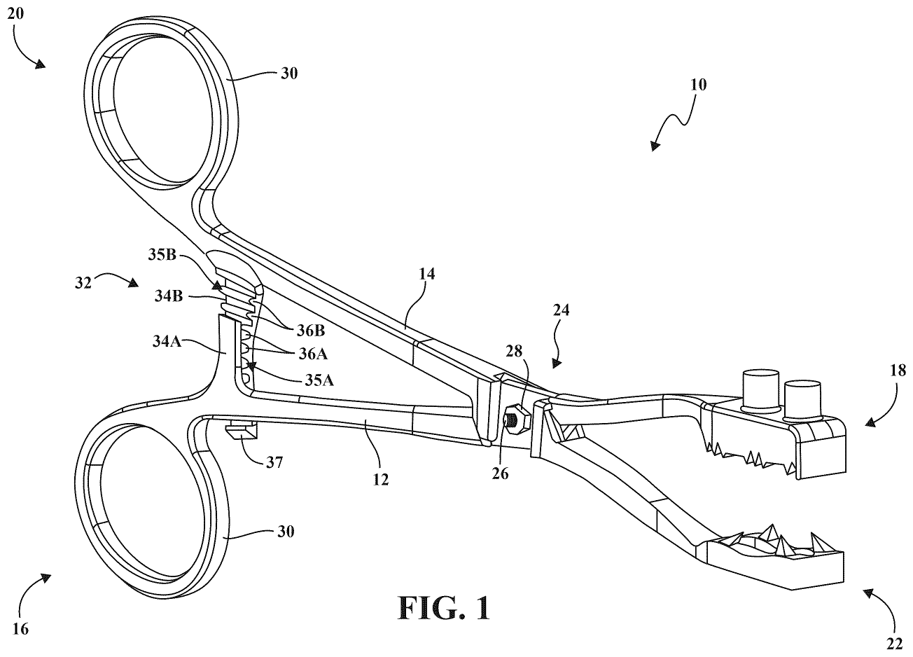

FIG. 1 illustrates a coracoid clamp 10 according to one exemplary aspect. The coracoid clamp 10 may include a first elongate arm 12 and a second elongate arm 14. The first elongate arm 12 may include a first proximal handle portion 16 and a first distal clamping portion 18. The second elongate arm 14 may include a second proximal handle portion 20 and a second distal clamping portion 22. As used herein, “proximal” may be understood to mean towards the practitioner holding the coracoid clamp 10, away from the surgical site to which the coracoid clamp 10 is applied, and “distal” may be understood to mean away from the practitioner, towards the surgical site and/or bone block to which the coracoid clamp 10 is applied.

The first elongate arm 12 may be pivotally coupled to the second elongate arm 14 by a pivot assembly 24 located between the first and second proximal handle portions 16, 20 and the first and second distal clamping portions 18, 22. The pivot assembly 24 may define a pivot point about which the first and second distal clamping portions 18, 22 are able to pivot towards one another to engage a bone block. More specifically, the pivot assembly 24 may be configured such that inward movement of the first and second proximal handle portions 16, 20 by a practitioner causes the first and second distal clamping portions 18, 22 to pivot towards one another in an inwards direction. Similarly, outward movement of the first and second proximal handle portions 16, 20 away from one another by the practitioner may cause the first and second distal clamping portions 18, 22 to pivot away from each other in an outwards direction. Each of the first and second proximal handle portions 16, 20 may include a finger hold 30 by which a practitioner may grasp and operate the coracoid clamp 10 with a single hand. As shown in the illustrated example, the pivot assembly 24 may be realized as a screw and nut mechanism, in which a screw 26 may be passed through aligned apertures in the first and second elongate arms 12, 14 and a nut 28 threaded onto the screw 26 to secure the pivot assembly 24 in place. According to some aspects, the screw 26 may include a portion of its length, namely a portion that sits within the aligned apertures of the first and second elongate arms 12, 14, that is not threaded in order to permit smoother rotation of the first and second elongate arms 12, 14 about the screw 26. In alternative aspects, the pivot assembly 24 may be realized by a hinge mechanism, ball-and-socket joint, or a rotating disc mechanism as non-limiting examples.

In some implementations, the coracoid clamp 10 may also include a ratcheting mechanism 32 extending between the first and second elongate arms 12, 14, such as between the first and second proximal handle portions 16, 20 and the pivot assembly 24. Ratcheting mechanism 32 may enhance the stability of the coracoid clamp 10 by helping to ensure that once the first and second distal clamping portions 18, 22 are engaged with a bone block, they remain securely in place, thereby minimizing any unintended movement or separation during the drilling process. Furthermore, the ratcheting mechanism 32 allows for incremental adjustments, enabling the practitioner to apply a precise amount of pressure to the bone block, which is helpful for maintaining the integrity and alignment of the bone block during drilling.

As shown in the illustrated example, the ratcheting mechanism 32 may be defined by a first protrusion 34A extending inwards from the first elongate arm 12 and a second protrusion 34B extending inwards from the second elongate arm 14 so as to overlap with one another between the first and second elongate arms 12, 14. The first and second protrusions 34A, 34B may each define a respective ratcheting side 35A, 35B that extends inwards from the corresponding first or second elongate arm 12, 14 and is configured to slide along the ratcheting side 35A, 35B of the other of the first and second protrusions 34A, 34B with the pivoting movement of the first and second proximal handle portions 16, 20 relative to one another. The ratcheting side 35A of the first protrusion 34A may define a series of teeth 36A that are distributed along a length of the first protrusion 34A defined by the inward extension of the first protrusion 34A. The teeth 36A may be configured to interact with a series of teeth 36B that are defined by the ratcheting side 35B of the second protrusion 34B and similarly distributed along a length of the second protrusion 34B defined by the inward extension of the second protrusion 34B. The interaction between the teeth 36A of the first protrusion 34A and teeth 36B of the second protrusion 34B may resist movement of the first and second proximal handle portions 16, 20 relative to each other, such as to help prevent inadvertent movement of the first and second distal clamping portions 18, 22 relative to one another.

In some implementations, the teeth 36A of the first protrusion 34A may be angled towards the first proximal handle portion 16 and the teeth 36B of the second protrusion 34B may be angled towards the second proximal handle portion 20. In this way, the first and second proximal handle portions 16, 20 may be moveable towards each other when a sufficient force is applied by the practitioner to the first and second proximal handle portions 16, 20 in the inward direction to overcome the resistance between the teeth 36A, 36B, and generally prevented from moving outwards away from each other absent one of the first and second protrusions 34A, 34B being flexed away from the other of the first and second protrusions 34B to disengage the teeth 36A from the teeth 36B, such as a via a finger tab 37 disposed at a free end of one of the first and second protrusions 34A, 34B that is opposite the end of the one of the first and second protrusion 34A, 34B coupled to the first or second elongate arm 12, 14.

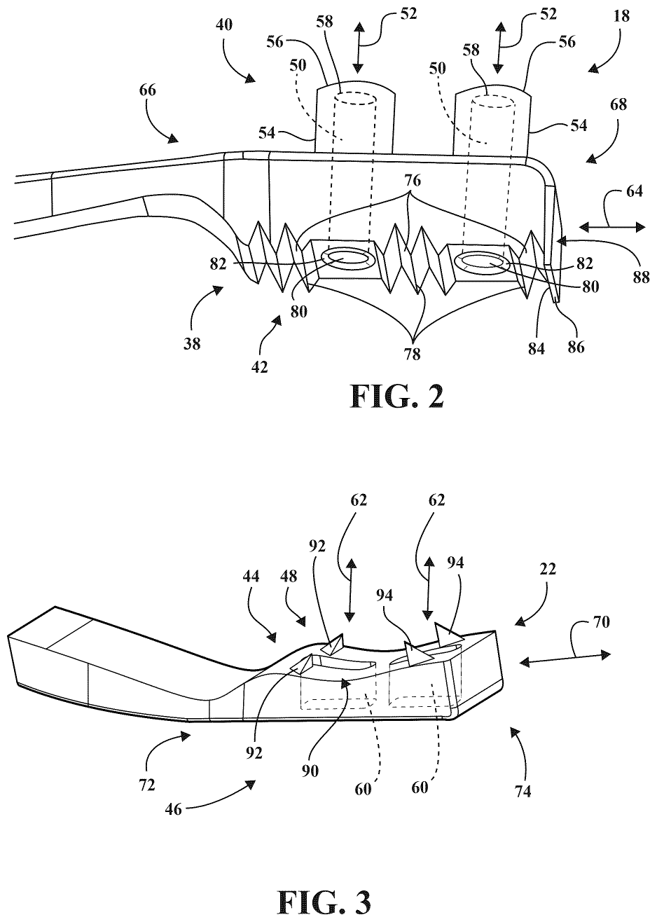

FIG. 2 illustrates the first distal clamping portion 18 according to an exemplary aspect. The first distal clamping portion 18 may include a first side 38 and a second side 40 opposite the first side 38. The first side 38 of the first distal clamping portion 18 may define a first bone block engagement structure 42. The first bone block engagement structure 42 may be specifically configured to engage a planar surface of a bone block, such as the prepared planar surface of the patient's coracoid. This engagement helps to stabilize the bone block by providing a flat surface against which to press by the first distal clamping portion 18.

FIG. 3 illustrates the second distal clamping portion 22 according to an exemplary aspect. The second distal clamping portion 22 may similarly include a first side 44 and a second side 46 opposite the first side 44. The first side 44 of the second distal clamping portion 22 may define a second bone block engagement structure 48. The second bone block engagement structure 48 may be specifically configured to engage a non-planar and/or naturally curved surface of the bone block. In other words, the second bone block engagement structure 48 may be designed to complement the natural anatomy of the coracoid, thereby ensuring a secure hold on the bone block by conforming to its curved surface.

As the first and second distal clamping portions 18, 22 are pivoted towards one another, the first sides 38, 44 may move inwards towards one another so as to engage a bone block with the first and second bone block engagement structures 42, 48. The first sides 38, 44 of the first and second distal clamping portions 18, 22 may thus be considered as inward-facing sides relative to movement the first and second distal clamping portions 18, 22, and the second sides 40, 46 of the first and second distal clamping portions 18, 22 may thus be considered as outward-facing sides relative to the movement of the first and second distal clamping portions 18, 22. The movement of the first and second distal clamping portions 18, 22 in the inwards direction may enable the first bone block engagement structure 42 to firmly engage the planar surface of the bone block while the second bone block engagement structure 48 engages the non-planar surface of the bone block. The first and second bone block engagement structures 42, 48 may thereby work in concert to securely hold the bone block in place, helping to prevent rotation and maintaining stability during the drilling process.

Referring again to FIG. 2, the first distal clamping portion 18 may define one or more lumens 50 each for guiding a drill bit into a different portion of a bone block engaged by the first and second bone block engagement structures 42, 48. Each lumen 50 may extend through the first bone block engagement structure 42 and the second side 40 of the first distal clamping portion 18. Each lumen 50 may also feature a circular cross-sectional shape in a direction transverse to the lumen's longitudinal axis 52 and configured to facilitate the precise drilling of holes. Specifically, the cross-sectional area of each lumen 50 may be sized so as to limit transverse movement of the drill bit within the lumen 50. The length of each lumen 50 along the lumen's longitudinal axis 52 may also be adequately sized so as to limit pivotal movement of the drill bit relative to the longitudinal axis 52 when disposed through the lumen 50.

For instance, in some implementations, the second side 40 of the first distal clamping portion 18 may include one or more tubes 54. Each tube 54 may extend in an outward direction relative to movement of the first distal clamping portion 18 and terminate at a free end 56 defining an aperture 58 to the lumen 50. Each of the tubes 54 may thus define a portion of one of the lumens 50 and function to extend the length of the lumen 50, helping to further ensure that the drill bit remains aligned with the longitudinal axis 52 of the lumen 50 throughout the drilling process.

Referring again to FIG. 3, the second distal clamping portion 22 may similarly define one or more lumens 60. Each lumen 60 may be configured for receiving a drill bit drilled bi-cortically through an engaged bone block via one of the lumens 50 of the first distal clamping portion 18, thus providing clearance for the drill bit after passing through the bone block. This clearance reduces the potential of the drill bit inadvertently contacting another object after drilling through the bone block, which can result in damage to the drill bit or undesired debris at a surgical site.

Each lumen 60 may extend through the second bone block engagement structure 48 and the second side 46 of the second distal clamping portion 22. Unlike each lumen 50 of the first distal clamping portion 18, each lumen 60 of the second bone block engagement structure 48 may have an elongated cross-sectional shape in a direction transverse to its longitudinal axis 62. In this way, each lumen 50 of the first distal clamping portion 18 may maintain alignment with a lumen 60 of the second distal clamping portion 22 such that the lumen 60 is positioned to receive a drill bit passed through the lumen 50 over a range of varying positions of the first distal clamping portion 18 relative to the second distal clamping portion 22. Correspondingly, the coracoid clamp 10 may be able to accommodate and guide the drilling of bone blocks of a range of shapes and sizes while also providing bi-cortical clearance for the drill bit.

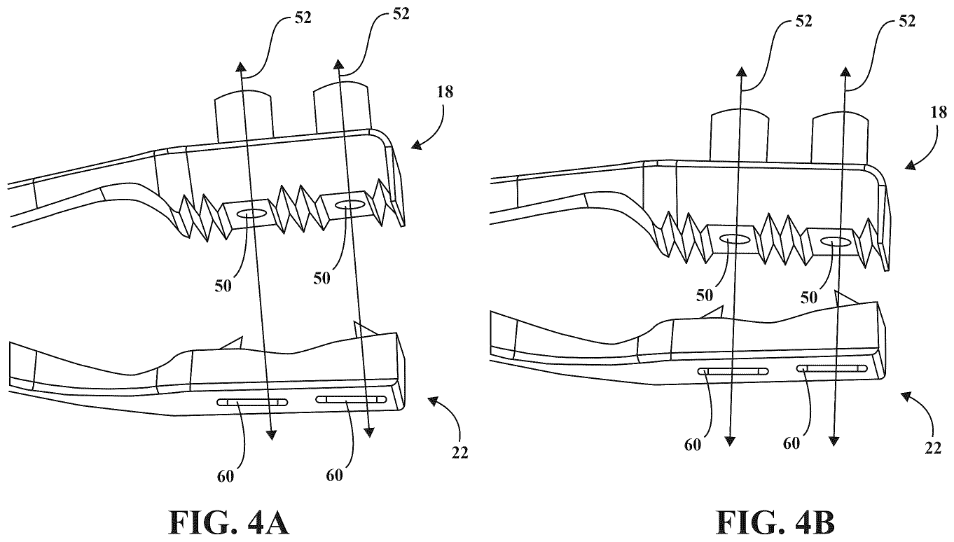

As an example, FIGS. 4A and 4B illustrate the first distal clamping portion 18 being moved into different positions relative to the second distal clamping portion 22. Specifically, the first and second distal clamping portions 18, 22 have been pivoted more inwards in FIG. 4B than in FIG. 4A. In both illustrations, the longitudinal axis 52 of each lumen 50 of the first distal clamping portion 18 extends through a corresponding lumen 60 of the second distal clamping portion 22. Consequently, in both positions, a drill bit passed through any one of the lumens 50 of the first distal clamping portion 18 and drilled through a bone block may be received in one of the lumens 60 of the second distal clamping portion 22.

Referring again to FIGS. 2 and 3, the first distal clamping portion 18 may have an elongated length along its longitudinal axis 64, which may form a portion of the central axis running the length of the first elongate arm 12. In other words, the longitudinal axis 64 of the first distal clamping portion 18 may extend through and between a proximal portion 66 and distal portion 68 of the first distal clamping portion 18. Similarly, the second distal clamping portion 22 may have an elongated length along its longitudinal axis 70, which may form a portion of the central axis running the length of the second elongate arm 14. In other words, the longitudinal axis 70 of the second distal clamping portion 22 may extend through and between a proximal portion 72 and distal portion 74 of the second distal clamping portion 22.

The first and second bone block engagement structures 42, 48 may be configured to engage a bone block such that the longitudinal axes 64, 70 of the first and second distal clamping portions 18, 22 run along a length of the coracoid, such as illustrated in FIG. 7 and described in more detail below. Assuming the first distal clamping portion 18 defines a plurality of lumens 50, the lumens 50 may be distributed along the longitudinal axis 64 of the first distal clamping portion 18 so as to guide the formation of drill holes in the bone block that are generally distributed along the length of the bone block. In other words, each lumen 50 of the first distal clamping portion 18 may be disposed between another one of the lumens 50 and either the proximal portion 66 or distal portion 68 of the first distal clamping portion 18.

Similarly, assuming the second distal clamping portion 22 defines a plurality of lumens 60, the lumens 60 may be distributed along the longitudinal axis 70 of the second distal clamping portion 22. In other words, each lumen 60 of the second distal clamping portion 22 may be disposed between another one of the lumens 60 and either the proximal portion 72 or distal portion 74 of the second distal clamping portion 22.

As shown in the illustrated example, in at least one aspect, the second distal clamping portion 22 may define a distinct lumen 60 for each lumen 50 of the first distal clamping portion 18. In at least one alternative aspect, the second distal clamping portion 22 may define a single lumen 60 for all of the lumens 50 of the first distal clamping portion 18, with the elongated cross-sectional dimension of the lumen 60 extending a majority of the length of the second distal clamping portion 22 so as to be configured to receive a drill bit passed through any of the lumens 50 of the first distal clamping portion 18.

As previously described, the first bone block engagement structure 42 may be configured to engage a planar surface of a bone block, such as a prepared surface of a coracoid, to help prevent rotation of the bone block during drilling. Referring again to FIG. 2, in one aspect, the first bone block engagement structure 42 may include a plurality of teeth 76. Each tooth 76 may extend in an inward direction relative to the pivotable movement of the first distal clamping portion 18 to define a free end 78 of the tooth 76. These free ends 78 may be configured to engage a planar surface of the bone block, helping to ensure a secure grip. According to some exemplary aspects, the free ends 78 of the teeth 76 may be substantially disposed in (e.g., within 1 mm of) a common plane that is transverse, or more particularly perpendicular, to the longitudinal axis 52 of each lumen 50. Alternatively, the free ends 78 of two or more (e.g., three) of the teeth 76 may be substantially disposed within the plane, while the free ends 78 of one or more other teeth 76 may not be substantially disposed within the plane. Each of the teeth 76 may extend laterally across the entire width of the first side 38 of the first distal clamping portion 18. The free ends 78 of the teeth 76 may bite into the planar surface of a bone block with movement of the first distal clamping portion 18 in the inward direction, helping to provide maximum contact with the planar surface of the bone block even in the presence of the slight deviations in the surface.

In a preferred configuration, the first bone block engagement structure 42 may include at least three teeth 76. The teeth 76 may be distributed along the elongated length of the first distal clamping portion 18, helping to ensure an even distribution of pressure across the planar surface of the bone block. For instance, at least one tooth 76 may be disposed between the distal portion 68 of the first distal clamping portion 18 and a distal-most lumen 50 of the first distal clamping portion 18, at least one tooth 75 may be disposed between the proximal portion 66 of the first distal clamping portion 18 and a proximal-most lumen 50 of the first distal clamping portion 18, and least one tooth 76 may be disposed between each pair of lumens 50 of the first distal clamping portion 18.

As shown in the illustrated example, in at least one aspect, the first side 38 of the first distal clamping portion 18 may define an aperture 80 to each lumen 50, and the teeth 76 of the first bone block engagement structure 42 may be spaced from each aperture 80 on both sides. Such spacing may help ensure that the drill bit does not contact the teeth 76 during insertion and removal of the drill bit through a lumen 50, which may result in damage to the drill bit and/or teeth 76, and may result in undesired debris at a surgical site. In at least one alternative aspect, the teeth 76 may be formed immediately up to and/or around each aperture 80. Regardless of the configuration of the teeth 76, in some aspects, the first side 38 of the first distal clamping portion 18 may also define a beveled surface 82 around each aperture 80, which may further help reduce potentially damaging contact between the drill bit and other objects such as the teeth 76.

Still referring to FIG. 2, the first side 38 of the first distal clamping portion 18 may also define a lip 84. The lip 84 may be configured to assist in positioning the first bone block engagement structure 42 relative to a planar surface of a bone block. The lip 84 may be positioned distal to the first bone block engagement structure 42, and may extend in the inward direction relative to the pivotable movement of the first distal clamping portion 18 and terminate at a free end 86. The free end 86 of the lip 84 may be located further inwards than the free ends 78 of the teeth 76. For instance, the lip 84 may extend through the common plane of the free ends 78 of the teeth 76 described above.

When engaging a planar surface of a bone block with the first bone block engagement structure 42, the lip 84 may be configured to be disposed against a side of the bone block extending from the planar surface and adjacent the conjoint tendon. Providing this reference point for engagement of the first bone block engagement structure 42 helps to ensure that the first bone block engagement structure 42 is positioned as desired relative to the bone block, reducing the risk of unsought drilling angles and/or positions. As shown in the illustrated example, in some implementations, the lip 84 may form a portion of a distal end surface 88 of the first distal clamping portion 18. Additionally or alternatively, the lip 84 may extend laterally across the entire width of first side 38 of the first distal clamping portion 18 to provide a wide contact surface for the reference point.

As previously described, the second bone block engagement structure 48 may be configured to engage a non-planar surface of the bone block opposite the planar surface engaged by the first bone block engagement structure 42. Referring again to FIG. 3, the second bone block engagement structure 48 may include a base 90 that faces inwards relative to movement of the second distal clamping portion 22, and that extends between the proximal portion 72 and the distal portion 74 of the second distal clamping portion 22. The second bone block engagement structure 48 may further include at least one proximal tooth 92 and at least one distal tooth 94 each extending inwards relative to the movement of the second distal clamping portion 22 from the base 90.

The base 90 may have a generally concave shape in the longitudinal direction of the second distal clamping portion 22. More specifically, the base 90 may be configured such that portions of the base 90 at the proximal and distal portions 72, 74 of the second distal clamping portion 22 are disposed further inwards than a portion of the base 90 between the proximal and distal portions 72, 74 of the second distal clamping portion 22. As shown in the illustrated example, the base 90 may be formed by an inward facing concave surface that extends between the proximal and distal portions 72, 74. According to alternative exemplary aspects, the base 90 may be formed by two or more inward facing planar surfaces distributed along the longitudinal axis 70 of the second distal clamping portion 22 and angled relative to each other to collectively form the base 90 with a generally concave shape. For instance, the base 90 may be formed by two inward facing planar surfaces generally forming a “V’ shape extending along the longitudinal axis 70 of the second distal clamping portion 22, by three inward facing planar surfaces that form a partial-hexagon shape extending along the longitudinal axis 70, and so on. The more planar surfaces that are used to form the base 90, the more the surfaces may collectively approximate a curve.

According to various exemplary aspects, the base 90 may also have a generally concave shape in two directions, namely the longitudinal direction as described above, and also in the lateral direction, such as to form the base 90 with a bowl-like shape. For instance, the bowl-like shape may be formed by a single inward facing continuous surface extending between the proximal and distal portions 72, 74 the second distal clamping portion 22, or alternatively by four or more inward facing planar surfaces angled relative to each other and arranged to form the bowl-like shape.

Each proximal tooth 92 of the second bone block engagement structure 48, which may form part of the proximal portion 72 of the second distal clamping portion 22, may be angled towards the distal portion 74 of the second distal clamping portion 22, and each distal tooth 94 of the second bone block engagement structure 48, which may form part of the distal portion 74 of the second distal clamping portion 22, may be angled towards the proximal portion 72 of the second distal clamping portion 22. Each proximal tooth 92 and distal tooth 94 may be pyramidally shaped to provide sharp points of engagement with the bone block.

As the second distal clamping portion 22 is moved to engage the non-planar surface of a bone block, the proximal and distal teeth 92, 94 may contact and slightly dig into the bone block, helping to provide a secure engagement with the non-planar surface that helps prevent slippage. The combination of the generally concave shape of the base 90 and the angled disposition of the proximal and distal teeth 92, 94 enables the second bone block engagement structure 48 to accommodate the varying natural curvature of different bone blocks. As shown in the illustrated example, the at least one proximal tooth 92 may include two proximal teeth 92 disposed at opposite corners of the proximal portion 72 of the second distal clamping portion 22, and the at least one distal tooth 94 may include two distal teeth 94 disposed at opposite corners of the distal portion 74 of the second distal clamping portion 22.

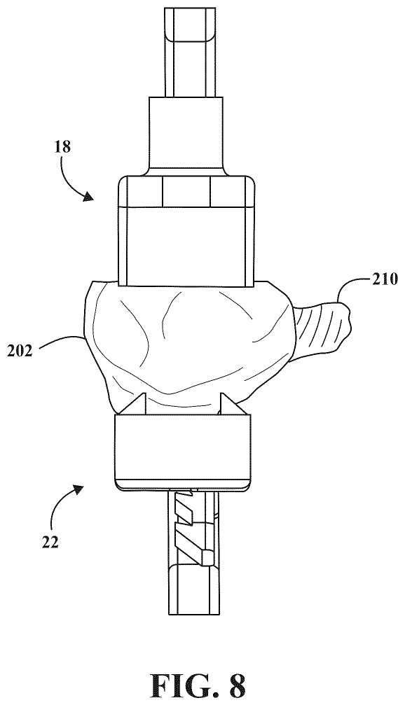

In some instances, when drilling holes through a bone block harvested from a patient's coracoid using the coracoid clamp 10, a surgeon may desire to keep a stump of the coraco-acromial ligament attached to the bone block for re-attachment to the shoulder capsule with sutures. To this end, the coracoid clamp 10 may be intentionally configured so that it does not fully encapsulate the bone block and allows space for this ligament to extend from either side of the coracoid clamp 10, such as to accommodate both right and left shoulder harvests. For instance, in at least some exemplary aspects, the lateral cross-section of the second distal clamping portion 22 between the proximal portion 72 and distal portion 74 may be shaped such that the portion of the cross-section forming the base 90 is linear (e.g., the cross-section may be rectangular) or convex. In this way, the coracoid clamp 10 may engage a bone block harvested from a patient's coracoid without interfering with a coraco-acromial ligament extending laterally from the bone block, such as illustrated in FIG. 8 and described in more detail below.

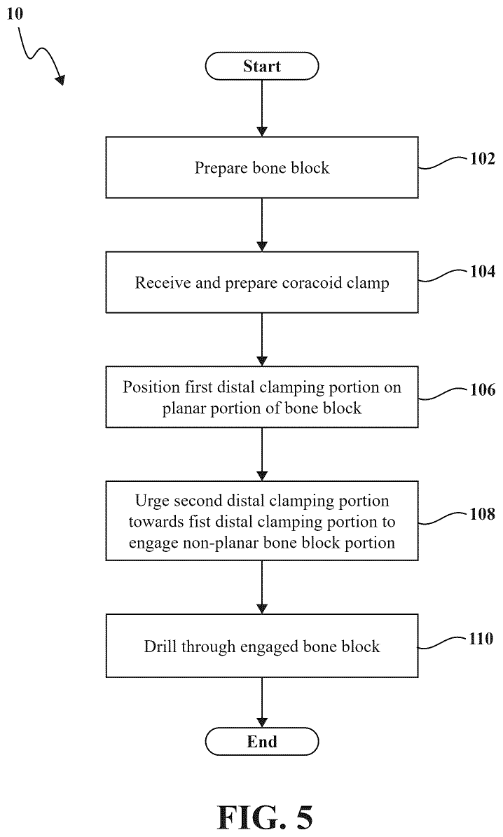

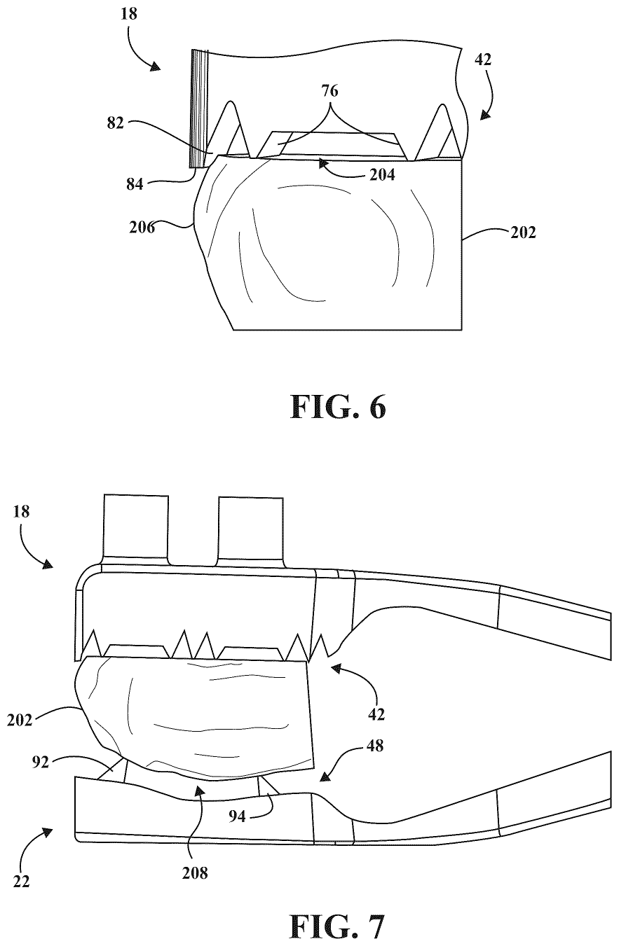

FIG. 5 illustrates a method 100 of preparing a bone block during a Latarjet procedure using the coracoid clamp 10, and FIGS. 6-9 illustrate performance of various steps of the method 100. In step 102, a bone block, such as for use in a Latarjet procedure, may be prepared for use with the coracoid clamp 10. For instance, a distal end portion of the patient's coracoid may be cut from the rest of the coracoid, and a planar surface may be prepared along a length of the cut portion to form the bone block. In step 104, the coracoid clamp 10 may be received and prepared for engagement with the bone block. For instance, a practitioner may grasp and move the proximal handle portions 16, 20 of the coracoid clamp 10 in the outward direction to cause a corresponding movement of the first and second distal clamping portions 18, 22.

In step 106, and as illustrated in FIG. 6, the first distal clamping portion 18 may be positioned against the bone block 202 such that the first bone block engagement structure 42 sits on the prepared planar surface 204 of the bone block 202, and such that the lip 84 is positioned against a side 206 of the bone block 202 that extends from the planar surface 204, such as may be adjacent to a conjoint tendon connected with the bone block 302 as described above.

In step 108, and as illustrated in FIG. 7, the second distal clamping portion 22 may be urged towards the first distal clamping portion 18 to engage a non-planar surface 208 of the bone block 202 with the second bone block engagement structure 48. Consequently, the bone block 202 may be held between the first bone block engagement structure 42 and the second bone block engagement structure 48. FIG. 8 illustrates another view of the bone block 202 being held between the first bone block engagement structure 42 and the second bone block engagement structure 48 as a result of step 108, with a stump 210 of the coracoacromial ligament still attached and extending laterally from the bone block 202 without disruption by the coracoid clamp 10, as described above.

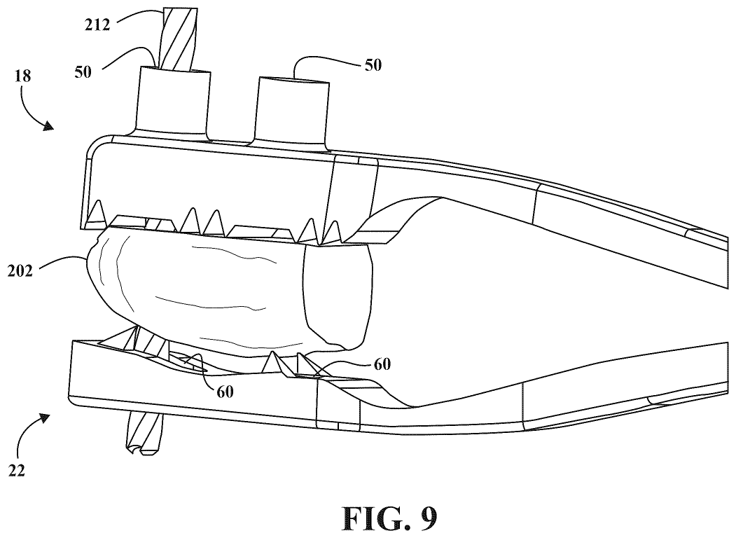

In step 110, and as illustrated in FIG. 9, a drill bit 212 may be passed through one or more of the lumens 50 of the first distal clamping portion 18 to form one or more holes in the bone block 202. As shown in FIG. 9, the drill bit 212 may be passed through a given lumen 50 and drilled bi-cortically through the bone block 202 so as to be received in a lumen 60 of the second distal clamping portion 22 aligned with the lumen 50.

A coracoid clamp 10 as described herein may be reusable, and may thus be formed of a material (e.g., stainless steel) that lends to reprocessing following a given procedure. Reprocessing of the coracoid clamp 10 according to one exemplary aspect may include disassembling the coracoid clamp 10 into its individual components (e.g., first and second elongate arms 12, 14, screw 26, nut 28), and then thoroughly cleaning and sterilizing each separate component. Cleaning may involve manual scrubbing, chemical cleaners, and/or the use of ultrasonic cleaners to remove any debris or contaminants. Sterilization may be performed following cleaning, and may include placing the components in an autoclave, which may employ high-pressure steam to eliminate any remaining microorganisms on the components. The components of the coracoid clamp 10 may then be reassembled as described herein in preparation for a next procedure. In some implementations, rather than being cleaned and/or sterilized, the removed screw 26 and nut 28 may be disposed of and replaced with a new screw 26 and a new nut 28 after the rest of the components of the coracoid clamp 10 have been cleaned and sterilized.

Implementations of a coracoid clamp as described herein offer several advantages over convention approaches including enhanced stability and precision during drilling. By incorporating features such as a bone block engagement structure specifically configured to engage a prepared flat surface of a bone block and an opposed bone block engagement structure with a concave surface and angled teeth that align with the natural curvature of the bone, the coracoid clamp is able to reduce the potential for slippage and rotation of engaged bone blocks of a wide range shapes and sizes during drilling, helping to ensure more accurate and reliable drill trajectories. The disclosed features may ultimately improve surgical outcomes by reducing the risk of non-union and bone cracking during the Latarjet procedure.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It will be understood that one or more steps within a method may be executed in a different order, may be executed serially or concurrently, and/or may include more or fewer steps than those illustrated and/or described above according to various aspects. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

### CLAUSES

I. A coracoid clamp comprising: a first elongate arm including a first proximal handle portion and a first distal clamping portion, the first distal clamping portion including a first side and a second side opposite the first side, the first side of the first distal clamping portion defining a first bone block engagement structure configured to engage a planar surface of a bone block; and a second elongate arm including a second proximal handle portion and a second distal clamping portion, the second distal clamping portion including a first side and a second side opposite the first side, the first side of the second distal clamping portion defining a second bone block engagement structure configured to engage a non-planar surface of the bone block opposite the planar surface, wherein the first elongate arm is pivotally coupled to the second elongate arm such that the first and second distal clamping portions are pivotable inwards towards each other with the first side of the first distal clamping portion defining an inward facing side of the first distal clamping portion and the second side of the first distal clamping portion defining an outward facing side of the first distal clamping portion, wherein the first distal clamping portion defines a lumen extending through the first bone block engagement structure and the second side of the first distal clamping portion, wherein the first bone block engagement structure comprises a plurality of first teeth each extending inwards to define a free end, the free ends of the first teeth being configured to engage the planar surface of the bone block, and wherein the first side of the first distal clamping portion defines a lip distal the first bone block engagement structure, the lip extending inwards to define a free end, the free end of the lip being located further inwards than the free end of each of the first teeth.

II. The coracoid clamp of claim I, wherein the lip forms a portion of a distal end surface of the first distal clamping portion.

III. The coracoid clamp of claim I or II, wherein the free ends of the first teeth are disposed in a common plane transverse to a longitudinal axis of the lumen.

IV. The coracoid clamp of claim III, wherein the common plane is perpendicular to the longitudinal axis of the lumen.

V. The coracoid clamp of any one of claims I-IV, where the first teeth comprise at least three teeth.

VI. The coracoid clamp of any one of claims I-V, wherein the lip and the first teeth each extends laterally across the first side of the first distal clamping portion.

VII. The coracoid clamp of any one of claims I-VI, wherein the second distal clamping portion comprises a proximal end and a distal end, and the second bone block engagement structure comprises a concave surface extending between the proximal end and the distal end of the second distal clamping portion, at least one second tooth extending from the concave surface and angled towards the proximal end of the second distal clamping portion, and at least one third tooth extending from the concave surface and angled towards the distal end of the second distal clamping portion.

VIII. The coracoid clamp of claim VII, wherein the at least one second tooth and the at least one third tooth are each pyramidally shaped.

IX. The coracoid clamp of any one of claims I-VIII, wherein the second distal clamping portion comprises a proximal portion, a distal portion, and an intermediate portion extending between the proximal and distal portions of the second distal clamping portion, the intermediate portion of the second distal clamping portion comprising a generally rectangular cross sectional shape in a direction transverse to a longitudinal axis of the second distal clamping portion that extends through the proximal portion and the distal portion of the second distal clamping portion.

X. The coracoid clamp of any one of claims I-IX, wherein the lumen has a circular cross sectional shape in a direction traverse to a longitudinal axis of the lumen.

XI. The coracoid clamp of any one of claims I-X, wherein the first distal clamping portion defines a plurality of lumens each extending through the first bone block engagement structure and the second side of the first distal clamping portion, a first of the lumens being disposed between a second of the lumens and the lip.

It will be further appreciated that the terms “include,” “includes,” and “including” have the same meaning as the terms “comprise,” “comprises,” and “comprising.” Moreover, it will be appreciated that terms such as “first,” “second,” “third,” and the like are used herein to differentiate certain structural features and components for the non-limiting, illustrative purposes of clarity and consistency. The terminology which has been used is intended to be in the nature of words of description rather than of limitation. Many modifications and variations are possible in light of the above teachings may be practiced otherwise than as specifically described.

What is claimed is:

1. A coracoid clamp comprising:
a first elongate arm including a first proximal handle portion and a first distal clamping portion, the first distal clamping portion including a first side and a second side opposite the first side, the first side of the first distal clamping portion defining a first bone block engagement structure configured to engage a planar surface of a bone block; and
a second elongate arm including a second proximal handle portion and a second distal clamping portion, the second distal clamping portion including a first side and a second side opposite the first side, the first side of the second distal clamping portion defining a second bone block engagement structure configured to engage a non-planar surface of the bone block opposite the planar surface,
wherein the first elongate arm is pivotally coupled to the second elongate arm such that the first and second distal clamping portions are pivotable towards each other to engage the bone block with the first bone block engagement structure and the second bone block engagement structure,
wherein the first distal clamping portion defines a plurality of first lumens each extending through the first bone block engagement structure and the second side of the first distal clamping portion, each of the first lumens having a circular cross sectional shape in a direction transverse to a longitudinal axis of the first lumen, and
wherein the second distal clamping portion defines a second lumen extending through the second bone block engagement structure and the second side of the second distal clamping portion, the second lumen having an elongated cross sectional shape in a direction transverse to a longitudinal axis of the second lumen.

2. The coracoid clamp of claim 1, wherein the first bone block engagement structure comprises a plurality of first teeth each defining a free end configured to engage the planar surface of the bone block.

3. The coracoid clamp of claim 2, wherein the free ends of the first teeth are disposed in a common plane transverse to the longitudinal axes of the first lumens.

4. The coracoid clamp of claim 3, wherein the common plane is perpendicular to the longitudinal axes of the first lumens.

5. The coracoid clamp of claim 4, where the first teeth comprise at least three teeth.

6. The coracoid clamp of claim 5, wherein the first distal clamping portion comprises a proximal end and a distal end, a first of the first teeth is disposed between a first of the first lumens and a second of the first lumens, a second of the first teeth is disposed between the first of the first lumens and the distal end of the first distal clamping portion, and a third of the first teeth is disposed between the second of the first lumens and the proximal end of the first distal clamping portion.

7. The coracoid clamp of claim 6, wherein each of the first teeth extends laterally across the first side of the first distal clamping portion.

8. The coracoid clamp of claim 7, wherein the first and second elongate arms are pivotable inwards towards each other such that the first side of the first distal clamping portion is further defined as an inward facing side of the first distal clamping portion, and the first side of the first distal clamping portion defines a lip distal to the first bone block engagement structure, the lip extending inwards to define a free end, the free end of the lip being located further inwards than the free end of each the first teeth.

9. The coracoid clamp of claim 8, wherein the lip forms a portion of a distal end surface of the first distal clamping portion.

10. The coracoid clamp of claim 9, wherein the lip extends laterally across the first side of the first distal clamping portion.

11. The coracoid clamp of claim 10, wherein the first distal clamping portion comprises a distal end, and a first of the first lumens is disposed between a second of the first lumens and the distal end of the first distal clamping portion.

12. The coracoid clamp of claim 11, wherein the second distal clamping portion defines a plurality of second lumens each extending through the second bone block engagement structure and the second side of the second distal clamping portion, each of the second lumens having an elongated cross sectional shape in a direction transverse to a longitudinal axis of the second lumen.

13. The coracoid clamp of claim 12, wherein the second distal clamping portion comprises a proximal end and a distal end, and the second bone block engagement structure comprises a concave surface extending between the proximal end and the distal end of the second distal clamping portion, at least one second tooth extending from the concave surface and angled towards the proximal end of the second distal clamping portion, and at least one third tooth extending from the concave surface and angled towards the distal end of the second distal clamping portion.

14. The coracoid clamp of claim 13, wherein the at least one second tooth and the at least one third tooth are each pyramidally shaped.

15. The coracoid clamp of claim 14, wherein the first elongate arm is pivotally coupled to the second elongated arm such that the second side of the first distal clamping portion is further defined as an outward facing side of the first distal clamping portion, and the second side of the first distal clamping portion comprises a plurality of tubes each extending outwardly to terminate at a free end of the tube and defining a portion of one of the first lumens.

16. The coracoid clamp of claim 15, wherein the second distal clamping portion comprises a proximal portion, a distal portion, and an intermediate portion extending between the proximal and distal portions of the second distal clamping portion, the intermediate portion of the second distal clamping portion comprising a generally rectangular cross sectional shape in a direction transverse to a longitudinal axis of the second distal clamping portion that extends through the proximal and distal portions of the second distal clamping portion.

17. The coracoid clamp of claim 16, wherein the first elongate arm is pivotally coupled to the second elongate arm at a pivot point located between the first and second proximal handle portions and the first and second distal clamping portions, the coracoid clamp comprising a ratcheting mechanism extending between the first and second elongate arms between the first and second proximal handle portions and the pivot point.

18. A method of preparing a bone block during a Latarjet procedure using a coracoid clamp, the method comprising:
providing a coracoid clamp comprising:
a first elongate arm including a first proximal handle portion and a first distal clamping portion, the first distal clamping portion including a first side and a second side opposite the first side, the first side of the first distal clamping portion defining a first bone block engagement structure configured to engage a planar surface of the bone block, and
a second elongate arm including a second proximal handle portion and a second distal clamping portion, the second distal clamping portion including a first side and a second side opposite the first side, the first side of the second distal clamping portion defining a second bone block engagement structure configured to engage a non-planar surface of the bone block that is opposite the planar surface,
wherein the first elongate arm is pivotally coupled to the second elongate arm such that the first and second distal clamping portions are pivotable towards each other, the first distal clamping portion defines a plurality of lumens each extending through the first bone block engagement structure and the second side of the first distal clamping portion, and the first side of the first distal clamping portion defines a lip distal to the first bone block engagement structure,
positioning the first distal clamping portion on a bone block such that the first bone block engagement structure sits on the planar surface of the bone block and the lip is positioned against a side of the bone block that extends from the planar surface and is adjacent a conjoint tendon;
engaging the coracoid clamp to urge the second distal clamping portion towards the first distal clamping portion to engage the non-planar surface of the bone block with the second bone block engagement structure and hold the bone block between the first bone block engagement structure and the second bone block engagement structure; and
drilling a hole into the held bone block through each of the lumens.

19. The method of claim 18, comprising engaging the coracoid clamp to urge the second distal clamping portion towards the first distal clamping portion to engage the bone block with the second bone block engagement structure and hold the bone block between the first bone block engagement structure and the second bone block engagement structure while a coracoacromial ligament extends laterally from the bone block.

20. A coracoid clamp comprising:
a first elongate arm including a first proximal handle portion and a first distal clamping portion, the first distal clamping portion including a first side and a second side opposite the first side, the first side of the first distal clamping portion defining a first bone block engagement structure configured to engage a planar surface of a bone block; and
a second elongate arm including a second proximal handle portion and a second distal clamping portion, the second distal clamping portion including a first side and a second side opposite the first side, the first side of the second distal clamping portion defining a second bone block engagement structure configured to engage a non-planar surface of the bone block opposite the planar surface,
wherein the first elongate arm is pivotally coupled to the second elongate arm such that the first and second distal clamping portions are pivotable inwards towards each other with the first side of the first distal clamping portion defining an inward facing side of the first distal clamping portion and the second side of the first distal clamping portion defining an outward facing side of the first distal clamping portion,
wherein the first distal clamping portion defines a lumen extending through the first bone block engagement structure and the second side of the first distal clamping portion,
wherein the first bone block engagement structure comprises a plurality of first teeth each extending inwards to define a free end, the free ends of the first teeth being configured to engage the planar surface of the bone block, and
wherein the first side of the first distal clamping portion defines a lip distal the first bone block engagement structure, the lip extending inwards to define a free end, the free end of the lip being located further inwards than the free end of each of the first teeth.
